(19) United States
(12) Patent Application Publication
(10) Pub. No.: US 2020/0179240 A1
(43) Pub. Date: Jun. 11, 2020

(54) NATURAL COSMETIC CLEANSER WHICH IS SOLID AT ROOM TEMPERATURE

(71) Applicant: Henkel AG & Co. KGaA, Duesseldorf (DE)

(72) Inventors: HEIKE SCHELGES, Willich (DE); ELVIRA SCHOLZ, Langenfeld (DE)

(73) Assignee: Henkel AG & Co. KGaA, Duesseldorf (DE)

(21) Appl. No.: 16/659,958

(22) Filed: Oct. 22, 2019

(30) Foreign Application Priority Data
Dec. 5, 2018 (DE) 10 2018 221 041.1

(51) Int. Cl. A61K 8/02; A61K 8/36; A61K 8/73; A61K 8/92; A61Q 5/02; A61Q 5/12

(52) U.S. Cl. CPC A61K 8/0216; A61K 8/361; A61K 8/737; A61K 2800/596; A61Q 5/02; A61Q 5/12; A61K 8/92

(57) ABSTRACT

In accordance with an exemplary embodiment, a cosmetic cleanser that is solid at about 25° C. includes—respectively with respect to its weight—from about 5% to about 90% by weight of an alkali salt of fatty acid (soap); from about 2% to about 50% by weight of an acyl glutamate as well as a natural polymer that has not been chemically modified.

### CROSS-REFERENCE TO RELATED APPLICATION

This application claims priority to German Patent Application No. 10 2018 221 041.1, filed Dec. 5, 2018, which is incorporated herein by reference in its entirety.

### TECHNICAL FIELD

The technical field relates to natural solid cleansers and conditioners for skin, and in particular for hair, which satisfy consumers' increasing desire for ecologically acceptable and natural products.

### BACKGROUND

Consumers are increasingly demanding cosmetic products that carry a natural cosmetics label and that are free from disputed ingredients. In order to provide the consumer with a guarantee that they are buying a natural product that is ecologically impeccable and safe, a huge number of certifying enterprises grant an appropriate seal of approval following individual testing.

This includes, inter alia, the BDIH label as an identifier for products that satisfy the strict requirements for genuine natural cosmetics. Currently, about 200 license holders and over 300 brands have been granted the BDIH label (Bundesverband der Industrie— and Handelsunternehmen für Arzneimittel, Reformwaren, Nahrungsergänzungs— and kosmetische Mittel, [Federal Association of German Industrial and Commercial Enterprises for Pharmaceuticals, Health Products, Food Supplements and Cosmetics], abbreviated to BDIH). Approximately 40 are located in Europe outside Germany and a further about 40 are outside Europe. In total, there are currently approximately 9000 products that have been awarded the BDIH seal.

As a supplement to the BDIH seal for controlled natural cosmetics, other seals have been established, for example the French “Ecocert” label. “Organic” has the same requirements for ingredients as another seal, the “NaTrue seal”. This is the same for “Made with organic ingredients”. For American products there is the “USDA seal”, and in England there is also the “Soil Association” label.

Since 2010, a further seal with the name “Cosmos” is becoming established for natural cosmetics and biocosmetics. The new label is binding for all innovations in natural cosmetics and biocosmetics. It covers all members of BDIH, Cosmebio, ICEA, Ecocert and the Soil Association. Overall, they represent the largest global system for the certification of bio-beauty products. Since that time, products from the members have been certified in accordance with the international Cosmos standard AISBL. Several thousand raw materials and also products have now been tested successfully. All results for natural cosmetics or biocosmetics that have been filed and tested since Jan. 1, 2017 then obtain the Cosmos certification from the respective member organization. Thus, the Cosmos-BDIH label or the Cosmos-Ecocert label is available.

A distinction is made between the inspection of natural cosmetics and the certification of bioproducts. The latter must additionally demonstrate that specified proportions of the ingredients are of biological origin. Thus, for a biocosmetic product, at least about 95% of the components obtained from plants must derive from an ecological agricultural source.

Furthermore, there is a growing trend for products that can dispense with plastic outer packaging. In this case, systems that are low in water such as soaps and syndets come to mind. In order to be able to guarantee both good performance on skin and hair along with simultaneous mildness, only a few foaming surfactants in powder form are available that also correspond to the Cosmos standard.

There is therefore a need for cosmetic cleansers and conditioners for skin, and in particular for hair, that are Cosmos certified and that can be marketed with a significantly reduced amount of packaging.

### BRIEF SUMMARY

In accordance with an exemplary embodiment, a cosmetic cleanser that is solid at about 25° C. is provided. The cosmetic cleanser comprises, respectively with respect to its weight,
a) from about 5% to about 90% by weight of an alkali salt of fatty acid (soap);
b) from about 2% to about 50% by weight of an acyl glutamate; and
c) a natural polymer that has not been chemically modified.

In accordance with another exemplary embodiment, a cosmetic cleanser that is solid at about 25° C. is provided. The cosmetic cleanser comprises, respectively with respect to its weight,
a) an alkali salt of fatty acid (soap) that is free from soap that is produced from palm oil;
b) sodium cocoyl glutamate; and
c) a natural polymer that has not been chemically modified selected from xanthan and guaran (INCI name: Guar Gum).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The objective of the various embodiments described herein solve the problems mentioned above. The objective forming the basis of the various embodiments is achieved by employing the subject matter of claim 1. In accordance with an exemplary embodiment, a cosmetic cleanser that is solid at about 25° C., contains—respectively with respect to its weight—
a) from about 5% to about 90% by weight of an alkali salt of fatty acid (soap);
b) from about 2% to about 50% by weight of an acyl glutamate; and
c) a natural polymer that has not been chemically modified.

The cleansers as contemplated herein are solid at about 25° C. “Solid materials” as contemplated herein means three-dimensional structures that have a stable shape and are not liquid or gaseous, i.e., retain their external shape even without a vessel surrounding it. However, the term “solid” is silent as regards the density or elasticity or other physical properties, so that gels, jellies, butters etc. may also be considered to be solid as contemplated herein, as long as they are stable in shape at about 25° C.

The cleansers as contemplated herein contain, as the first ingredient, one or more soap(s) in a total quantity of all alkali salts of fatty acids of from about 5% to about 90% by weight. Particularly preferably, the soap(s) are used within narrow ranges of quantities. In this case, preferred cosmetic cleansers as contemplated herein contain from about 10% to about 89% by weight, preferably from about 20% to about 88% by weight, more preferably from about 30% to about 87% by weight, yet more preferably from about 40% to about 86% by weight and in particular from about 50% to about 85% by weight of alkali salts of fatty acids (soaps).

Fatty acids are aliphatic carboxylic acids with formula R¹CO—OH, in which R¹CO represents an aliphatic, linear or branched acyl residue containing 6 to 22 carbon atoms and 0 and/or 1, 2 or 3 double bonds. Typical examples are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucaic acid, as well as their technical mixtures which, for example, precipitate out during the hydrolysis of natural fats and oils under pressure. Technical fatty acids containing 12 to 18 carbon atoms such as, for example, coconut, palm, palm kernel or tallow fatty acid, are preferred, wherein as contemplated herein, the use of coconut fatty acids is particularly preferred and the use of raw materials from palm oil should be dispensed with.

Particularly preferred cleansers as contemplated herein are free from soaps that are produced from palm oil.

Sodium salts are particularly preferred alkali salts because of their application-related properties in the context herein. Particularly preferred cosmetic cleansers as contemplated herein contain from about 10% to about 89% by weight, preferably from about 20% to about 88% by weight, more preferably from about 30% to about 87% by weight, yet more preferably from about 40% to about 86% by weight and in particular from about 50% to about 85% by weight of sodium salts of C₁₂₋₁₈ fatty acids.

As the second ingredient, the cleansers as contemplated herein contain 2% to 50% by weight of acyl glutamate(s). These compounds are produced from L-glutamic acid and fatty acids that can be found in nature and are characterized by low foam formation and good washing properties.

Particularly preferably, the cosmetic cleansers as contemplated herein contain from about 3% to about 40% by weight, preferably from about 4% to about 35% by weight, more preferably from about 5% to about 30% by weight, yet more preferably from about 6% to about 25% by weight and in particular from about 7.5% to about 15% by weight of acyl glutamates with formula (I)

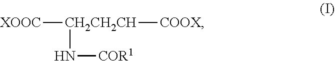

in which R¹CO represents a linear or branched acyl residue containing 6 to 22 carbon atoms and 0 and/or 1, 2 or 3 double bonds and X represents hydrogen, an alkali and/or an alkaline-earth metal, ammonium, alkylammonium, alkanolammonium or glucammonium.

Again with a view to the possibility of certification as a natural cosmetic, in the context herein, it is preferable to use alkali metal ions and in particular sodium ions as the cation X. Regarding the fatty residues, as was the case with the soaps, the use of coconut fatty acids is particularly preferred, and the use of raw materials from palm oil should be dispensed with.

Particularly preferred cleansers are free from acyl glutamates that are produced from palm oil.

More particularly preferred cosmetic cleansers as contemplated herein contain an acyl glutamate in which X represents Na and R¹CO represents an acyl residue that is derived from coconut oil (INCI: Sodium Cocoyl Glutamate).

In addition to the soap(s) and the acyl glutamate(s), the cleansers as contemplated herein may additionally contain further surfactants. Having regard to the problems mentioned above and the compatibility of the cleansers with the skin, it has been shown to be advantageous if only surfactants from specific groups of materials are used. Having regard to consumer acceptance, skin compatibility and application properties, cosmetic cleansers as contemplated herein that exclusively contain surfactants from the groups formed by:
alkyl oligosaccharides and polysaccharides; and
betaines
   are particularly preferred.

Particularly preferably, the cosmetic cleansers as contemplated herein—with respect to their weight—contain less than about 1% by weight, preferably less than about 0.5% by weight, more preferably less than about 0.5% by weight and in particular less than about 0.1% by weight of sulfate-containing surfactant(s), wherein most preferred cleansers are completely free from sulfate-containing surfactants.

The optional preferred surfactants that may be used are described below.

Alkyl and alkenyl oligoglycosides constitute known non-ionic surfactants that can be described by the formula

R¹O—[G]ₚ

in which R¹ represents an alkyl and/or alkenyl residue containing 4 to 22 carbon atoms, G represents a sugar residue containing 5 or 6 carbon atoms and p represents numbers from about 1 to about 10. They can be obtained using relevant preparative organic chemistry methods.

The alkyl oligoglycosides and/or alkenyl oligoglycosides may be derived from aldoses or ketoses containing 5 or 6 carbon atoms, preferably from glucose. The preferred alkyl oligoglycosides and/or alkenyl oligoglycosides are thus alkyl- and/or alkenyl oligoglucosides. The index number p in the general formula gives the degree of oligomerization (DP), i.e. the distribution of monoglycosides and oligoglycosides and represents a number from about 1 to about 10. While p must always be a whole number in the compound given and above all can take the value p=from about 1 to about 6 in this case, the value p for a specific alkyl oligoglycoside is an analytically determined mathematical factor which usually represents a fractional number. Preferably, alkyl oligoglycosides and/or alkenyl oligoglycosides with a mean degree of oligomerization p of from about 1.1 to about 3.0 are used. From an application-related viewpoint, such alkyl oligoglycosides and/or alkenyl oligoglycosides are preferred that have a degree of oligomerization of less than about 1.7 and in particular from about 1.2 to about 1.4. The alkyl residue or alkenyl residue R¹ may be derived from primary alcohols containing 4 to 11, preferably 8 to 10 carbon atoms. Typical examples are butanol, caproic alcohol, caprylic alcohol, capric alcohol and undecyl alcohol as well as their technical mixtures as obtained, for example, by the hydrolysis of technical fatty acid methyl esters or during the hydrolysis of aldehydes in the Roelen oxosynthesis process. Alkyl oligoglucosides with a chain length of C₈-C₁₀ (DP=from about 1 to about 3), which arise as a precursor in the distillative separation of technical C₈-C₁₈ coconut oil alcohol and which may be contaminated with a proportion of less than 6% by weight of C₁₂ alcohol, as well as alkyl oligoglucosides based on technical C_{9/11} oxo alcohols (DP=from about 1 to about 3) are also contemplated herein. The alkyl or alkenyl residue R¹ may also be derived from primary alcohols containing 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinic alcohol, arachidyl alcohol, gadoleyl alcohol, behenyl alcohol, erucyl alcohol, brassidyl alcohol as well as their technical mixtures, which may be obtained as described above. Alkyl oligoglucosides based on hardened C_{12/14} coconut alcohol with a DP of from about 1 to about 3 are preferred.

Preferred cleansers as contemplated herein contain from about 0.1% to about 10% by weight, preferably from about 0.25% to about 7.5% by weight, more preferably from about 0.5% to about 5% by weight, yet more preferably from about 0.75% to about 2.5% by weight and in particular from about 1 to about 2% by weight of surfactant(s) from the group formed by alkyl oligosaccharides and alkyl polysaccharides.

Betaines are known surfactants that are primarily produced by carboxyalkylation, preferably carboxymethylation of amine compounds. Preferably, the starting materials are condensed with halocarboxylic acids or their salts, in particular with sodium chloroacetate, wherein one mole of salt is formed per mole of betaine. Furthermore, the deposition of unsaturated carboxylic acids such as acrylic acid, for example, is also possible. Examples of suitable betaines are the carboxyalkylation products of secondary amines and in particular tertiary amines, which may be described by the formula:

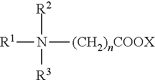

in which R¹ represents alkyl residues and/or alkenyl residues containing 6 to 22 carbon atoms, R² represents hydrogen or alkyl residues containing 1 to 4 carbon atoms, R³ represents alkyl residues containing 1 to 4 carbon atoms, n represents numbers from 1 to 6 and X represents an alkali and/or alkaline-earth metal or ammonium. Typical examples are carboxymethylation products of hexylmethylamine, hexyldimethylamine, octyldimethylamine, decyldimethylamine, dodecylmethylamine, dodecyldimethylamine, dodecylethylmethylamine, C_{12/14} Coco alkyldimethylamine, myristyldimethylamine, cetyldimethylamine, stearyldimethylamine, stearylethylmethylamine, oleyldimethylamine, and C_{16/18} tallow alkyldimethylamine, as well as their technical mixtures.

Furthermore, carboxyalkylation products of amidoamines described by the following formula:

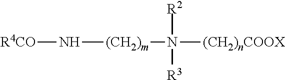

may also be considered, in which R⁴CO represents an aliphatic acyl residue containing 6 to 22 carbon atoms and 0 or from about 1 to about 3 double bonds, m represents numbers from about 1 to about 3 and R², R³, n and X have the meanings given above. Typical examples are reaction products of fatty acids containing 6 to 22 carbon atoms, namely caproic acid, caprylic acid, capric acid, lauric acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucaic acid as well as their technical mixtures, with N,N-dimethylaminoethylamine, N,N-dimethylaminopropylamine, N,N-diethylaminoethylamine and N,N-diethylaminopropylamine, which are condensed with sodium chloroacetate. Preferably, a condensation product of C_{8/18} coconut fatty acid-N,N-dimethylaminopropylamide with sodium chloroacetate is used.

Furthermore, suitable starting materials for the betaines used as contemplated herein that may also be considered are imidazolines, which can be described by the formula:

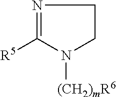

in which R⁵ represents an alkyl residue containing 5 to 21 carbon atoms, R⁶ represents a hydroxy group, an OCOR⁵ or NHCOR⁵ residue and m represents from about 2 or about 3. These substances too are known substances that, for example, may be obtained by cyclization condensation of about 1 or about 2 moles of fatty acid with multivalent amines such as, for example, aminoethyl ethanolamine (AEEA) or diethylene triamine. The corresponding carboxyalkylation products constitute mixtures of different open-chained betaines. Typical examples are condensation products of the fatty acids cited above with AEEA, preferably imidazolines based on lauric acid or, in fact C_{12/14} coconut fatty acid, which are then turned into betaines with sodium chloroacetate.

Preferred cleansers as contemplated herein contain from about 0.5% to about 15% by weight, preferably from about 1% to about 10% by weight, more preferably from about 1.5% to about 7.5% by weight, yet more preferably from about 2% to about 6% by weight and in particular from about 2.5% to about 5% by weight of surfactant(s) from the betaine group.

The cleansers as contemplated herein contain, as the third ingredient, at least one natural polymer that has not been chemically modified. Substances of this type, also termed biopolymers, derive in particular from the polysaccharide group. The use of cellulose, starch, guar gum or xanthan is particularly preferred.

The term “that has not been chemically modified” means that the natural polymer has not undergone any chemical reactions in order to modify its properties. In contrast, “physical modifications” are possible, and are common with starches and xanthan, for example (pregelatinization, cooking, cold swelling or instantized starches, heat-treated xanthan). Physically modified starches are equated with native starches because they have only been heat-treated, i.e. cooked.

Independently of whether only one or more natural polymer(s) are used, preferred cosmetic cleansers as contemplated herein contain from about 0.1% to about 10% by weight, preferably from about 0.25% to about 9% by weight, more preferably from about 0.5% to about 8% by weight, yet more preferably from about 0.75% to about 7% by weight and in particular from about 1% to about 6% by weight of natural, non-modified polymer(s).

Particularly preferably, the cosmetic cleansers as contemplated herein contain from about 0. I% to about 5% by weight of guaran (INCI name Guar Gum).

Guaran, also known as guar gum, is a vegetable gum. The chemical bond from the polysaccharide group is the major component of guar germ meal (or guar meal for short). Guaran includes D-mannopyranose units which are bonded together via β-1,4-glycosidic bonds. In addition, alternate mannopyranose units carry α-D-galactopyranosyl residues via a 1,6-bond.

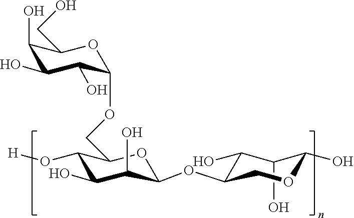

Preferably, the guar gum is used in narrower ranges of quantities. Here, preferred cleansers as contemplated herein contain from about 0.2% to about 4% by weight, preferably from about 0.3% to about 3.5% by weight, more preferably from about 0.4% to about 3% by weight, yet more preferably from about 0.45% to about 2.5% by weight and in particular from about 0.5% to about 2% by weight of guaran.

The substances as contemplated herein may preferably contain from about 0.1% to about 5% by weight of xanthan (INCI name Xanthan Gum). Xanthan gum is a natural, sustainable raw material and is excreted as an anionic polysaccharide from the bacterium Xanthomonas campestris.

The molecular weight of the xanthan gum used is preferably about from 2×10⁶ to about 20×10⁶ g/mol.

Xanthan gum contains D-glucose, D-mannose, D-glucuronic acid, acetate and pyruvate as molecular components in an approximate molar ratio of about 28 to about 30 to about 20 to about 17 to about 5.1 to about 6.3. The polymeric backbone of the xanthan gum is formed by a cellulose chain formed from β-1,4-bonded glucose units. Xanthan contains structural units with the following formula:

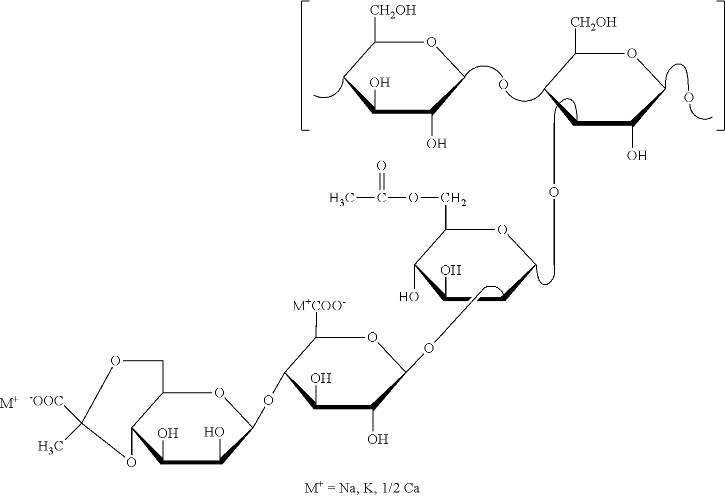

If appropriate, heat-treated xanthan gum may also be used in the cosmetic cleansers as contemplated herein.

In a preferred embodiment, the cleanser as contemplated herein contains, as the xanthan gum:
heat-treated xanthan gum, or
a mixture of xanthan gum and heat-treated xanthan gum.

When using a mixture of xanthan gum and heat-treated xanthan gum, it has been shown to be particularly effective for the xanthan gum and the heat-treated xanthan gum to be used in a weight ratio range of from about 2 to about 1 to about 1 to about 20, in particular of from about 1 to about 2 to about 1 to about 10.

The term “heat-treated xanthan gum” as contemplated herein should be understood to mean xanthan gum that is subjected to at least 40° C. of heat. The resulting heat-treated xanthan gum has an improved dispersibility and can be dispersed faster in water than xanthan gum that has not undergone heat treatment. In a 1% by weight aqueous solution, the preferred suitable heat-treated xanthan gum has a viscosity of at least from about 25000 to about 40000 mPa·s (Brookfield DV-I viscosimeter, spindle #6 at about 23° C. and about 10 rpm). Preferred heat-treated xanthan gums that may be used produce a pH of from about 4.0 to about 6.0 at about 23° C. when in about 1% by weight aqueous solution.

The heat-treated xanthan gum that is preferred as contemplated herein was obtained by tempering xanthan gum at a temperature of at least about 60° C., in particular at least about 100° C. Tempering may be carried out using a multitude of known methods such as, for example, by heat treatment using an oven, fluidized bed, infrared or microwave. In the context of the above heat treatments, it is furthermore preferable for the xanthan gum to have a water content of less than about 25% by weight, in particular of less than about 8% by weight, more particularly preferably of less than about 3% by weight prior to the heat treatment. More preferably as contemplated herein, the cleanser has therein such heat-treated xanthan gum that has been obtained by heat treating xanthan gum with a water content of less than about 25% by weight at a temperature of at least about 60° C. (in particular at least about 100° C.) for at least about 30 minutes. Particularly preferably as contemplated herein, the cleanser employs such heat-treated xanthan gum wherein xanthan gum with a water content of less than about 8% by weight has been heat-treated at a temperature of at least about 60° C. (in particular at least about 100° C.) for at least about 30 minutes. The preferred duration of said heat treatments of the xanthan gum - in particular with said water content—at a temperature of at least about 60° C. (in particular at least about 100° C.) is at least about 1 hour. The particularly preferred duration of said heat treatments of the xanthan gum—in particular with the preferred water content—at a temperature of at least about 60° C. (in particular at least about 100° C.) is at least about 2.5 hours.

Preferably—irrespective of whether the xanthan gum is heat-treated xanthan or non-heat-treated xanthan or comprises a mixture of both—it is used in a narrow range of quantities. In this case, preferred cleansers as contemplated herein contain from about 0.1% to about 10% by weight, preferably from about 0.25% to about 9% by weight, more preferably from about 0.5% to about 8% by weight, yet more preferably from about 0.75% to about 7% by weight and in particular from about 1% to about 6% by weight of xanthan.

The cleansers as contemplated herein may contain from about 0.1% to about 10% by weight of betaine (N,N,N-trimethylammonioacetate or N,N,N-trimethylglycine). Preferably, the betaine is used within narrow ranges of quantities and also from native sources. In this case, preferred cosmetic cleansers as contemplated herein contain from about 0.1% to about 10% by weight, preferably from about 0.2 to about 9% by weight, more preferably from about 0.3% to about 8% by weight, yet more preferably from about 0.4% to about 7% by weight, yet more preferably from about 0.45% to about 6% by weight and in particular from about 0.5% to about 5% by weight of natural betaine.

They contain less than about 25% by weight, preferably less than about 20% by weight, more preferably less than about 15% by weight, yet more preferably less than about 10% by weight and in particular from about 0.1 to about 5% by weight of water.

Particularly preferably, the cleansers as contemplated herein may contain further components, wherein in particular, natural skin care and moisturizing substances are suitable. Particularly preferably, native oils are used, so that preferred cosmetic cleansers contain from about 0.1% to about 10% by weight, preferably from about 0.25% to about 9% by weight, more preferably from about 0.5% to about 8% by weight, yet more preferably from about 0.75% to about 7% by weight and in particular from about 1% to about 6% by weight of natural oil(s).

Particularly preferably, the cleansers as contemplated herein contain one or more of the following cited oils in a total oil quantity as defined above: acacia oil, algae oil, argan oil (from the fruit of the argan tree), avocado oil (from the flesh of the avocado fruit from the avocado tree), babassu oil, cottonseed oil (from the seeds of the cotton plant), borage oil or borage seed oil (from the seeds of the borage plant), cupuacu butter, cashew shell oil, thistle oil (also known as “safflower oil”, from the seeds of the safflower or carthamus), peanut oil (from the fruit of the peanut plant), hazelnut oil (from hazelnuts from the hazelnut bush), hemp oil (from the seeds of edible hemp), jatropha oil (from the seeds of jatropha curcas), jojoba oil (actually a liquid wax; from the seeds of the jojoba shrub), camomile oil (from the seeds of camellia oleifera, camellia sinensis or camellia japonica), cocoa butter, coconut oil (from the seeds of the coconut, the fruit of the coconut palm tree), pumpkin seed oil (also known as pepita oil; from the seeds of the Styrian oil squash), linseed oil (from ripe linseeds from flax), camelina oil (from the seeds of gold-of-pleasure, Brassicaceae family), macadamia oil (from the nuts of the macadamia tree), corn oil (from corn seeds), almond oil (from almonds from the almond tree), mango butter (from mangifera indica), apricot kernel oil (from the apricot kernel—i.e. the pit of the apricot kernel—from the apricot), poppyseed oil (from the seeds of the poppy), evening primrose oil, olive oil (from the fruit and kernel of the olive, the fruit from the olive tree), palm oil (from the fruit of the palm fruit, the fruit of the oil palm), palm kernel oil (from the kernel of the palm fruit, the fruit of the oil palm), papaya oil, pistachio oil, pecan nut oil, perilla oil from the seeds of the perilla plant (shiso, Korean perilla), rapeseed oil (from the seeds from rape, Brassicaceae family), rice oil, castor oil (from the seed of the miracle tree), sallow thorn oil (from the fruit of sallow thorn berries, the fruit from the sallow thorn bush), sallow thorn kernel oil (from the kernels from sallow thorn berries, the fruit from the sallow thorn bush), mustard oil (from the black mustard seeds), black cumin oil (from the seeds of the fruit capsule from the black cumin plant), sesame oil (from the seeds of the sesame plant), shea butter (from the seeds of the shea nut tree), soya oil (from soybeans), sunflower oil (from sunflower seeds), tung oil, walnut oil (from the kernels from the nuts from the walnut tree), watermelon seed oil, grapeseed oil (from the kernels from the fruit (grapes) of the grape vine or vine), wheatgerm oil (from wheatgerm), and cedarwood oil (from the wood from cedar of Lebanon).

The cleansers as contemplated herein may contain further ingredients; within the spirit and scope of the various embodiments, care should be taken that COSMOS certified raw materials are employed.

### EXAMPLES

The following compositions may be produced by way of example (all details are as a % by weight):

| | 01 | 02 | 03 | 04 | 05 | 06 | 07 | 08 | 09 |
|---|---|---|---|---|---|---|---|---|---|
| Sodium Sunflowerseedate | 60.23 | 60.23 | 60.23 | 60.23 | 60.23 | 60.23 | 60.23 | 60.23 | 60.23 |
| Sodium Cocoate | 12.83 | 12.83 | 12.83 | 12.83 | 12.83 | 12.83 | 12.83 | 12.83 | 12.83 |
| Sodium Cocoyl Glutamate | 5.00 | 5.00 | 5.00 | 5.00 | 7.00 | 3.00 | 5.00 | 5.00 | 5.00 |
| Cocamidopropyl Betaine | 4.19 | 4.19 | 4.19 | 4.19 | 3.00 | 7.00 | 4.19 | 4.19 | 4.19 |
| Glycerin | 2.57 | 2.57 | 2.57 | 2.57 | 2.57 | 2.57 | 2.57 | 2.57 | 2.57 |
| Cocos Nucifera | 2.00 | 2.00 | 2.00 | 2.00 | 2.00 | 2.00 | 3.00 | 5.00 | 5.00 |
| (Coconut) Oil | | | | | | | | | |
| Sodium Chloride | 1.59 | 1.59 | 1.59 | 1.59 | 1.59 | 1.59 | 1.59 | 1.59 | 1.59 |
| Citric Acid | 0.43 | 0.43 | 0.43 | 0.43 | 0.43 | 0.43 | 0.43 | 0.43 | 0.43 |
| Xanthan | 1.00 | 2.00 | 0.50 | 3.00 | 1.00 | 1.00 | 1.00 | 1.00 | 3.00 |
| Tetrasodium Glutamate | 0.05 | 0.05 | 0.05 | 0.05 | 0.05 | 0.05 | 0.05 | 0.05 | 0.05 |
| Diacetate | | | | | | | | | |
| Fragrance | 1.00 | 1.00 | 1.00 | 1.00 | 1.00 | 1.00 | 1.00 | 1.00 | 1.00 |
| Aqua (Water, Eau) | ad 100 | | | | | | | | |
| | 10 | 11 | 12 | 13 | 14 | 15 | 16 | 17 | 18 |
| Sodium Palmate | 56.49 | 56.49 | 56.49 | 56.49 | 56.49 | 56.49 | 56.49 | 56.49 | 56.49 |
| Sodium Palm Kernelate | 14.36 | 14.36 | 14.36 | 14.36 | 14.36 | 14.36 | 14.36 | 14.36 | 14.36 |
| Sodium Cocoyl Glutamate | 5.00 | 5.00 | 5.00 | 5.00 | 7.00 | 3.00 | 5.00 | 5.00 | 5.00 |
| Cocamidopropyl Betaine | 4.19 | 4.19 | 4.19 | 4.19 | 3.00 | 7.00 | 4.19 | 4.19 | 4.19 |
| Glycerin | 3.96 | 3.96 | 3.96 | 3.96 | 3.96 | 3.96 | 3.96 | 3.96 | 3.96 |
| Cocos Nucifera | 2.00 | 2.00 | 2.00 | 2.00 | 2.00 | 2.00 | 3.00 | 5.00 | 5.00 |
| (Coconut) Oil | | | | | | | | | |
| Sodium Chloride | 1.21 | 1.59 | 1.59 | 1.59 | 1.59 | 1.59 | 1.59 | 1.59 | 1.59 |
| Sodium Hydroxide | 0.04 | 0.05 | 0.05 | 0.05 | 0.05 | 0.05 | 0.05 | 0.05 | 0.05 |
| Xanthan | 1.00 | 2.00 | 0.50 | 3.00 | 1.00 | 1.00 | 1.00 | 1.00 | 3.00 |
| Tetrasodium Glutamate | 0.05 | 0.43 | 0.43 | 0.43 | 0.43 | 0.43 | 0.43 | 0.43 | 0.43 |
| Diacetate | | | | | | | | | |
| Fragrance | 1.00 | 1.00 | 1.00 | 1.00 | 1.00 | 1.00 | 1.00 | 1.00 | 1.00 |
| Aqua (Water, Eau) | ad 100 | | | | | | | | |
| | 19 | 20 | 21 | 22 | 23 | 24 | 25 | 26 | 27 |
| Sodium Olivate | 75.19 | 75.19 | 75.19 | 75.19 | 75.19 | 75.19 | 75.19 | 75.19 | 75.19 |
| Sodium Cocoyl Glutamate | 5.00 | 5.00 | 5.00 | 5.00 | 7.00 | 3.00 | 5.00 | 5.00 | 5.00 |
| Cocamidopropyl Betaine | 4.19 | 4.19 | 4.19 | 4.19 | 3.00 | 7.00 | 4.19 | 4.19 | 4.19 |
| Glycerin | 0.86 | 0.86 | 0.86 | 0.86 | 0.86 | 0.86 | 0.86 | 0.86 | 0.86 |
| Cocos Nucifera | 2.00 | 2.00 | 2.00 | 2.00 | 2.00 | 2.00 | 3.00 | 5.00 | 5.00 |
| (Coconut) Oil | | | | | | | | | |
| Sodium Chloride | 1.00 | 2.00 | 0.50 | 3.00 | 1.00 | 1.00 | 1.00 | 1.00 | 3.00 |
| Xanthan | 5.00 | 5.00 | 5.00 | 5.00 | 7.00 | 3.00 | 5.00 | 5.00 | 5.00 |
| Tetrasodium Glutamate | 0.05 | 0.05 | 0.05 | 0.05 | 0.05 | 0.05 | 0.05 | 0.05 | 0.05 |
| Diacetate | | | | | | | | | |
| Fragrance | 1.00 | 1.00 | 1.00 | 1.00 | 1.00 | 1.00 | 1.00 | 1.00 | 1.00 |

| | |
|---|---|
| Aqua (Water, Eau) | ad 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic cleanser that is solid at about 25° C. comprising, respectively with respect to its weight,
d) from about 5% to about 90% by weight of an alkali salt of fatty acid (soap);
e) from about 2% to about 50% by weight of an acyl glutamate; and
f) a natural polymer that has not been chemically modified.

2. The cosmetic cleanser as claimed in claim 1, comprising from about 10% to about 89% by weight of alkali salts of fatty acids (soaps), based on the total weight of the cosmetic cleanser.

3. The cosmetic cleanser as claimed in claim 2, wherein the alkali salts of fatty acids (soaps) comprise sodium salts of C₁₂₋₁₈ fatty acids.

4. The cosmetic cleanser as claimed in claim 1, comprising, based on the total weight of the cosmetic cleanser, from about 3% to about 40% by weight of acyl glutamates with formula (I) 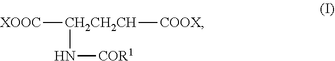
in which R¹CO represents a linear or branched acyl residue comprising 6 to 22 carbon atoms and 0 and/or 1, 2 or 3 double bonds and X represents hydrogen, an alkali and/or alkaline-earth metal, ammonium, alkylammonium, alkanolammonium or glucammonium.

5. The cosmetic cleanser as claimed in claim 4, wherein the acyl glutamates comprise an acyl glutamate in which X represents Na and R¹CO represents an acyl residue that is derived from coconut oil (INCI: Sodium Cocoyl Glutamate).

6. The cosmetic cleanser as claimed in claim 1, comprising, based on the total weight of the cosmetic cleanser, from about 0.1% to about 10% by weight of natural polymer(s) that have not been modified.

7. The cosmetic cleanser as claimed in claim 6, wherein the natural polymer(s) comprises xanthan.

8. The cosmetic cleanser as claimed in claim 1, comprising less than about 25% by weight of water.

9. The cosmetic cleanser as claimed in claim 1, comprising from about 0.1% to about 10% by weight of natural oil(s), based on the total weight of the cosmetic cleanser.

10. The cosmetic cleanser as claimed in claim 1, comprising - with respect to its total weight—less than about 1% by weight of sulfate-containing surfactant(s).

11. The cosmetic cleanser as claimed in claim 10, wherein the cosmetic cleanser if free from sulfate-containing surfactants.

12. The cosmetic cleanser as claimed in claim 1, wherein the cosmetic cleanser is free from soaps that are produced from palm oil.

13. The cosmetic cleanser as claimed in claim 1, wherein the cosmetic cleanser is free from acyl glutamates that are produced from palm oil.

14. The cosmetic cleanser as claimed in claim 1, further comprising a surfactant selected from the group comprising alkyl oligosaccharides, alkyl polysaccharides, and betaines.

15. The cosmetic cleanser as claimed in claim 14, comprising from about 0.1% to about 10% by weight of a surfactant from the group of alkyl oligosaccharides and alkyl polysaccharides, based on the total weight of the cosmetic cleanser.

16. The cosmetic cleanser as claimed in claim 14, comprising from about 0.5% to about 15% by weight of a surfactant comprising a betaine, based on the total weight of the cosmetic cleanser.

17. The cosmetic cleanser as claimed in claim 6, comprising from about 0.1% to about 5% by weight of guaran (INCI name Guar Gum), based on the total weight of the cosmetic cleanser.

18. A cosmetic cleanser that is solid at about 25° C. comprising, respectively with respect to its weight,
d) an alkali salt of fatty acid (soap) that is free from soap that is produced from palm oil;
e) sodium cocoyl glutamate; and
f) a natural polymer that has not been chemically modified selected from xanthan and guaran (INCI name: Guar Gum).

19. The cosmetic cleanser as claimed in claim 18, further comprising further comprising a surfactant selected from the group comprising alkyl oligosaccharides, alkyl polysaccharides, and betaines.

20. The cosmetic cleanser as claimed in claim 18, wherein the alkali salt of fatty acids (soap) is present in an amount of from about 5% to about 90% by weight and the sodium cocoyl glutamate is present in an amount of from about 2% to about 50% by weight, based on the total weight of the cosmetic cleanser.
